(19) United States
(12) Patent Application Publication
(10) Pub. No.: US 2010/0015635 A1
(43) Pub. Date: Jan. 21, 2010

(54) IMMOBILISATION OF FLUORESCENT PROTEINS

(76) Inventors: Gerard W. Canters, Leiden (NL); Gerhild Zauner, Leiden (NL); Armand W. J. W. Tepper, Leiden (NL); Luigi Bubacco, Padua (IT); Thijs Aartsma, Leiden (NL); Gerhild Zauner, Leiden (NL)

Correspondence Address: WHITHAM, CURTIS & CHRISTOFFERSON, P.C., 11491 SUNSET HILLS ROAD, SUITE 340, RESTON, VA, 20190, US

(21) Appl. No.: 12/442,261

(22) PCT Filed: Sep. 21, 2007
(86) PCT No.: PCT/EP2007/060065

§ 371 Date: Sep. 4, 2009

(30) Foreign Application Priority Data
Sep. 21, 2006 (EP) 06121062.1
Aug. 24, 2007 (GB) 0716634.1

(51) Int. Cl. G01N 33/573; G01N 33/00

(52) U.S. Cl. 435/7.4; 436/86

(57) ABSTRACT

The present invention concerns a method of detection of an analyte in which a protein capable of binding the analyte and comprising a fluorescent energy label and an energy acceptor moiety capable of accepting energy emitted by the label or protein by Forster energy transfer (FRET), is exposed to incident electromagnetic energy to excite the protein or label, and the fluorescent emission of the label is measured; characterised in that the protein is encapsulated in a biocompatible, optically transparent matrix which is permeable to the analyte, and in that the protein undergoes no substantial conformational change during the method; further characterised in that the energy acceptor moiety has a more active and less active state, which is determined by the presence of analyte, and the emission from the label is indicative of the presence of analyte. A biocompatible optically transparent matrix in which a protein capable of binding an analyte is also provided.

### STATEMENT OF THE INVENTION

The present invention relates to methods of detection in which fluorescent emission from a labelled protein is measured. The protein is encapsulated in a biocompatible, optically transparent polymer matrix. The fluorescent emission can be related to the concentration of an analyte present. Typically, the analyte is oxygen.

### BACKGROUND TO THE INVENTION

The mechanism underlying the present invention is fluorescent resonance energy transfer (FRET). FRET occurs between a donor and an acceptor moiety.

FRET is based on a distance-dependent interaction between the electronic excited states of two dye molecules in which excitation is transferred from a donor molecule to an acceptor molecule without the emission of a photon. This process is known as Förster energy transfer. The efficiency of FRET is dependent on the inverse sixth power of intermolecular separation [1], making it useful over distances comparable with the dimensions of biological macromolecules. When FRET is used as a contrast mechanism, colocalisation of proteins and other molecules can be imaged with spatial resolution beyond the limits of conventional optical microscopy [2].

In order for FRET to occur the donor and acceptor molecules must be in close proximity (typically 10-100\acute{Å}), the absorption spectrum of the acceptor must overlap with the fluorescence emission spectrum of the donor, and the donor and acceptor transition dipole vectors must be approximately parallel, or at least not orthogonal.

Existing methods of oxygen detection are based often on (bulky) solid state sensors, the prototype of which is the Clark electrode [3]. One of the Clark electrode's drawbacks is that it consumes oxygen during measurement, thereby changing the oxygen concentration in the sample. Other sensors are based on measuring the luminescence of an oxygen sensitive reporter molecule [4], usually a ruthenium compound. Such a compound has the drawback of being unstable in water and therefore requiring encapsulation in a hydrophobic matrix. Application of both types of sensors is further limited because of possible interference by other substances and because of relatively long response times.

We have described in a previous patent application, WO2006/066977, how FRET can be used to monitor the activity of a donor-acceptor pair on a protein. In that application, a labelled protein comprising a fluorescent donor label and an energy acceptor moiety is subjected to incident radiation to excite the donor and the fluorescence emission of the donor is measured. The emission intensity varies with the level of quenching by the acceptor moiety, which typically depends upon its oxidation state.

Erker et al [5] have described how fluorescent labels may be used as sensors for the oxygen binding of arthropod hemocyanins. FRET from fluorescent labels to oxygenated active sites quenches the emission of the labels by roughly 50% upon oxygenation of the protein.

In the past decade, the encapsulation of enzymes inside inorganic sol-gel matrices has been used to prepare efficient biocatalysts which are easy to recycle. Pierre et al in [6] provide a detailed overview of the use of sol-gel processes for enzyme encapsulation. The review mentions that in optical biosensors, the enzyme must be labelled with fluorescent or chromophoric groups. Fluorescent quenching techniques are mentioned, but only in the context of the study of conformation modifications.

Gadre et al in [7] described the use of biodoped ceramics for use in the encapsulation of biologicals such as proteins. The sol-gel technique, using metal alkoxide precursors such as tetraethyl orthosilicate and tetramethyl orthosilicate, is described.

In sol-gels, precursor compounds are used to build a silica network. The silica network may encapsulate a protein, such as an enzyme. The two most commonly used precursors are:

1) Alkoxide TetraMethOxySilane (TMOS), which is by far the best studied and the most used among a variety of precursors now available [6]. The resulting sol-gels may be used in biosensors [8].

2) Sodium metasilicate (Na₂SiO₃), commonly termed waterglass, which has as its main advantage that no alcohols (which could be harmful for the biomolecule) are generated as byproducts during the encapsulation step [6, 9].

### SUMMARY OF THE INVENTION

In accordance with a first aspect of the invention, there is provided a method of detection of an analyte in which a protein capable of binding the analyte and comprising a fluorescent energy label and an energy acceptor moiety capable of accepting energy emitted by the label or protein by Förster energy transfer (FRET), is exposed to incident electromagnetic energy to excite the protein or label, and the fluorescent emission of the label is measured;

characterised in that the protein is encapsulated in a biocompatible, optically transparent matrix which is permeable to the analyte, and in that the protein undergoes no substantial conformational change during the method;

wherein the energy acceptor moiety has a more active and less active state, which is determined by the presence of analyte, and the emission from the label is indicative of the presence of analyte.

In accordance with a second aspect of the invention, there is provided a biocompatible, optically transparent matrix in which a protein capable of binding an analyte is encapsulated, wherein the matrix is permeable to the analyte and the protein comprises a fluorescent energy label and an acceptor moiety capable of accepting energy emitted by the label or protein by Förster energy transfer, wherein the energy acceptor moiety has a more and a less active state between which the moiety can be converted.

A third aspect of the invention is a method of making an electrode for detecting an analyte, in which a coating comprising a matrix and a protein is coated onto an electrode substrate, wherein the matrix is as defined in the second aspect of the invention.

In the invention, the emission from the label is indicative of the presence of analyte.

This may be interpreted in the sense
a) that the label fluorescence (after excitation either directly or by the intrinsic fluorescence from the protein) is quenched by direct FRET from the label to the acceptor moiety;
b) that the label fluorescence is not quenched but that the intensity of the excitation source (for instance, the intrinsic fluorescence from the protein) is quenched by FRET to the acceptor moiety; or
c) a combination of a) and b) (for instance, in the case that the intrinsic fluorescence of the protein is used to excite the label).

Case c) mentioned above has the particular advantage that it results in a gain in sensitivity: the intrinsic fluorescence of the protein is (partly) quenched and, in addition, the label fluorescence is (partly) quenched. Both of these quenching effects are the result of FRET to the acceptor moiety. The effects are cumulative and result in enhanced quenching. For instance, if the intrinsic protein fluorescence is quenched to 70% of its original fluorescence, and the label fluorescence quenches to 60%, the fluorescence of the label will exhibit an overall reduction to 42% of its original fluorescence level.

By a proper choice of the fluorescent label the present invention provides both a sensitive and selective method for the detection of an analyte. Since FRET efficiency depends on the inverse sixth power of the distance between emitter and absorber, energy transfer from other molecules is not efficient enough to compete with the intra-molecular energy transfer to the label. The advantage of measuring the sensitised fluorescence in particular is that the signal is free from background noise. Since the label can be chosen so that the emission occurs in the visible range of the spectrum where there is no interference from other emitting species in the solution, the method is selective. Moreover, depending on the efficiency of energy transfer, there may also be a gain in quantum yield of the fluorescence when comparing the sensitized fluorescence with the intrinsic fluorescence of the protein. This also helps to increase the sensitivity of the method proposed herein.

Immobilisation has the potential to provide a more stable and, above all, reusable sensor. The matrix can be selected to provide an appropriate environment for the protein, which allows it to maintain its native structure, spectroscopic properties and catalytic properties upon encapsulation into the matrix.

### DETAILED DESCRIPTION OF THE INVENTION

In the method, the protein does not undergo a substantial conformational change. There should be substantially no change in the distance between the donor (protein or label) and acceptor moiety. By this we mean that the distance between the donor and the acceptor does not alter enough to significantly affect the FRET between the donor and the acceptor. Typically, analyte binding affects the donor-acceptor distance by less than 10%, preferably less than 5%, more preferably less than 1%.

In a first embodiment of this invention, the method of detection involves the use of a protein comprising a fluorescent energy donor label and at least one energy acceptor moiety capable of accepting the energy from the label by Förster energy transfer, as described in WO2006/066977. In such an embodiment of the invention, the incident electromagnetic energy excites the fluorescent energy donor label, and emission from the label is reduced when the energy acceptor moiety is in its more active state.

In this embodiment the incident electromagnetic energy should have a wavelength in the range 400 to 700 nm.

The activity of the or each energy acceptor moiety is related to its ability to accept the energy from the donor label and quench the donor's fluorescent emission. It therefore follows that the more active state accepts energy more readily than the less active state and consequently quenches more of the donor's fluorescence. In a preferred embodiment of the invention the less active energy acceptor state is completely inactive and will therefore quench no donor fluorescence. This facilitates experimental detection of the state of the energy acceptor moiety.

Typically, the binding of the analyte to the moiety converts the moiety to its more active state and reduces the donor fluorescence.

In a second embodiment of the invention, the incident electromagnetic energy does not excite the label, but rather excites amino acid residues in the protein, or a cofactor. This typically induces intrinsic emission from the protein. This intrinsic emission can induce emission from the label, in a process known as “sensitised fluorescence”. However, energy can also be transferred to the acceptor moiety, thereby reducing the emission from the label. This allows detection of an analyte, since binding of the analyte changes the state of the acceptor moiety, and its ability to accept energy by FRET.

For this embodiment of the present invention to allow detection of an analyte, FRET must be possible between the fluorescent protein residues or cofactor and the fluorescent label, i.e. the emission spectra of the fluorescent protein residues should overlap with the absorption spectrum of the fluorescent label and the residues and label must be in sufficiently close proximity for FRET to occur. In addition, the absorption spectrum of either the bound or unbound acceptor moiety of the protein must overlap with the emission spectra of the fluorescent protein residues to allow a change in emission from the fluorescent label when an analyte binds to the protein.

In this embodiment, preferably, the fluorescent label absorbs radiation in the wavelength range 330-450 nm, preferably most strongly at about 350 nm, and fluoresces in the visible range of the spectrum, i.e. 400-700 nm. Fluorescence of the label in the visible region of the spectrum advantageously reduces the interference or ‘noise’ from fluorescence of protein residues. Suitable fluorescent labels include Cy5, Atto390, Atto655, Alexa350 and Cy3.

The incident radiation should be suitable for exciting the protein to induce intrinsic fluorescence therefrom. Preferably, the incident radiation has a wavelength of 260-450 nm, more preferably 280-300 nm if Trp residues are to be excited.

Typical energy acceptor moiety-fluorescent label distances are in the range 1-4 nm. This is typically around the Förster distance. However, for this second embodiment of the present invention, the distance from the fluorescent label to the energy acceptor moiety is less important than the distance from the label to the source of endogenous fluorescence (usually the Trp residues). Proteins usually contain several Trp residues and these should be within the Förster distance of the energy acceptor moiety for a quenching effect to be observed.

Typically, the binding of the analyte to the energy acceptor moiety reduces the amount of intrinsic emission from the protein converted through FRET into emission from the label. The binding of the analyte may allow a second FRET channel to be opened up for the excited protein residues to the acceptor moiety, consequently reducing the energy channeled to the label and inducing a drop in label fluorescence. For this to occur, the absorption spectrum of the moiety when bound to the analyte should overlap with the intrinsic emission from the protein. The concept is illustrated further in FIG. 1.

As detailed above, when excited by UV light, proteins often exhibit a conspicuous fluorescence that originates from aromatic residues. Typically, this fluorescence, or “intrinsic emission” from the protein is due to the tryptophan residues in the protein. Alternatively, phenylalanine or tyrosine residues, or an organic cofactor may fluoresce.

In the method of the present invention, the emission from the fluorescent label is typically not “all or nothing” (within a bulk solution) and may vary proportionally with the concentration of analyte in the medium suspected of containing the analyte. This advantageously allows the concentration of the analyte to be determined. When observing single molecules, the fluorescence is typically on or off (or high or low). The concentration of the analyte in the single molecule case is then reflected by the average number of the on and off periods per unit time.

The or each energy acceptor moiety of the labelled protein in this invention may be reversibly converted from its more active state to its less active energy state and vice versa. This may occur by a chemical/biochemical reaction or a change in the environmental conditions surrounding the acceptor molecule. For example, an enzymatic reaction may occur which alters the energy-absorbing ability of the acceptor moiety. Suitable enzymes include proteases, kinases, phosphatases, glycosylases, glycosidases, oxido-reductases and transferases. Alternatively, a pH change in the external medium may switch the energy acceptor from its more to its less active form.

The or each energy acceptor may also be non-reversibly converted between its more and less active states. This would be of use in an assay where a one-off experiment is sufficient.

It is envisaged that the invention will have utility wherein the protein is an enzyme, preferably a redox enzyme. The analyte may be a (co-)substrate, inhibitor or cofactor of the enzyme. A (co-)substrate in this specification means one of two or more substrates of an enzyme. The analyte is typically a molecule which binds to the protein and causes the energy acceptor moiety to be converted between its more and its less active states. The analyte typically associates with the moiety through non-covalent interactions. The dissociation constant for an analyte may vary widely, spanning a range from nM to mM.

The analyte may be converted to another species by the enzyme, or alternatively may bind reversibly, as in the case of some enzyme inhibitors. In some embodiments of the invention, the analyte binds the moiety reversibly and is chemically identical before and after binding.

The moiety typically binds the analyte. Typically the moiety is a chromophore, the absorption of which changes as a result of enzymatic activity, or analyte binding. Enzymes typically contain metal ions, metal ion complexes comprising two or more metal ions (preferably transition metal ions) and organic cofactors (such as flavin) for binding of external molecules. Copper, iron and nickel ions are particularly preferred metal ions. Suitable organic cofactors include orthoquinone and pyridoxal-5-phosphate. Any of these may constitute the moiety of the protein used in the present invention.

A suitable moiety is Cu₂, as in tyrosinase. Alternatively, the moiety may be Cu₃, as in laccase. An analyte (for example oxygen) may reversibly bind to Cu₂. Alternatively, the analyte may bind to the moiety and be converted to product. When O₂ binds to a Cu₃ centre, for example, the centre is (partly) reduced and the O₂ is converted to peroxide or water. An inhibitor of an enzyme comprising a Cu₃ centre, on the other hand, may bind reversibly to the centre.

Preferably, the protein is an oxygen carrier, oxygenase or an oxidase. The oxygen carrier may be hemocyanin (Hc) from an anthropod or mollusc, or alternatively haemerythrin. Suitable oxygenases catalyse the hydroxylation of phenols and the oxidation of the diphenol products to the corresponding quinones (for example, tyrosinase, Ty). The oxygenase enzyme may be a monooxygenase. Suitable oxidases may convert o-diphenols to the corresponding quinones (for example, catechol oxidase, CO).

Other proteins which may have utility in the present invention include laccase, polyphenol oxidases, cytochrome P450 enzymes, hydrogenases and ureases. Preferably, the protein is a metalloprotein. The metalloprotein may belong to the family of blue copper proteins, or be a conjugate of one or more of these proteins, giving a fusion protein. Members of this family include copper-containing laccases and oxidases and the small blue copper proteins, for example azurin, from Pseudomonas aeruginos, pseudoazurin from Alcaligenes faecalis, plastocyanin from Fem Dryopteris crassirhizoma and amicyanin from Paracoccus versutus. Haem containing proteins like cytochrome c550 from P. versutus and flavin-containing proteins like flavadoxin 11 from A. vinelandii may also be used in the present invention. Furthermore, the method may be used with redox enzymes, for example, methylamine dehydrogenase (MADH) from Paracoccus denitrificans, nitrite reductase (NiR) from Alcaligenes faecalis, tyrosinase and Small Laccase (SLAC) from Streptomyces coelicolor.

Azurin is a 14 kDa extensively studied protein carrying a single copper ion at its redox active centre. In its oxidised (Cu²⁺) form the protein displays a strong (ε, absorption coefficient=5.6 mM⁻¹cm⁻¹) absorption in the 550-650 nm range, which corresponds to a π-π* transition of the Cu site, involving mainly the d_{x2-y2} orbital on the Cu and a 3p orbital on the Cys112 sulfur. This absorption disappears when the Cu site is reduced because in the reduced (Cu⁺) form the Cu has a d¹⁰ electronic configuration and the optical absorption spectrum lacks conspicuous features (ε<10M⁻¹,cm⁻¹).

This pronounced change of absorption spectrum will strongly modulate the fluorescence properties of a FRET donor-acceptor pair, with the Cu-site as the energy acceptor and a dye-label, suitably linked to the protein, as the fluorescence donor. When Cu is in the oxidised state the fluorescence of the dye is strongly quenched as a result of the energy transfer to the π-π* excited state of the Cu site (which is non-fluorescing itself), whereas with the Cu in the reduced state the fluorescence is essentially uninhibited since the π-π* transition is absent. Thus the fluorescent dye acts as a passive “beacon” which is off (i.e. quenched) in the oxidised and on (not quenched) when Cu is in the reduced state in the protein.

Preferably, when the protein is a metalloprotein, the method of the present invention also involves physiological partner proteins. In this embodiment the labelled protein docks with, for instance, a redox partner protein to/from which it donates or accepts electrons. The partner protein converts the energy acceptor moiety between its two states. The substrate of the enzyme may be the analyte to be detected. The redox partner protein may be an enzyme capable of oxidising or reducing substrates where upon the labelled protein is switched between its states. The level of quenching in this case is indicative of the extent of the enzymic redox reaction and may be used to detect the presence or level of substrate. Table 1 lists a selection of systems which can be studied using the method of the present invention involving redox partner proteins.

TABLE 1

| Enzyme | Protein Partner | Detects |
|---|---|---|
| Nitrite reductase | (Pseudo)azurin, (p)Az | Nitrite (NO₂⁻/NO) |
| Cytochrome p450 | Flavodoxin (FLD) | Aromatic compounds |
| Methylamine | Amicyanin | Methylamine |
| dehydrogenase | | |
| Cytochrome c550 | Amicyanin | Various |

The partners of amicyanin are methylamine dehydrogenase (MADH) and cytochrome c550. The cyt-c550 functions as an electron shuttle and passes the electrons it receives from amicyanin on to other members of the electron transfer chain, i.e., respiratory enzymes like the membrane bound aa₃ cytochrome oxidase. The function of cyt c550 resembles that of amicyanin in that it accepts and passes on electrons.

Mutants of the wild-type proteins included within the scope of the present invention may also be prepared. These are useful to extend the range of analytes which may be detected. The mutants may be engineered using a directed evolution approach based on random PCR and a new screening procedure based on the fluorescence detection of NADPH consumption by P450 BM3 in whole E. coli cells.

Either pAz or NiR can be labelled with a suitable fluorophore at a position on the protein surface. Upon excitation of the label fluorescence quenching would take place when the type 1 Cu site is in the oxidised (Cu(II)) state, but would not take place when the Cu is reduced. The change in the fluorescence signal may be used to monitor the transfer of electrons between the partner proteins. No change is to be expected in the absence of analyte (NO₂⁻ in this case).

Since Förster transfer depends on an overlap of the fluorescence spectrum of the donor with the acceptor, it can be calculated (see Example 2 of WO2006/066977) that the Förster radius (the distance at which FRET is 50% efficient—i.e. half of the donors are deactivated) of the oxidised type 1 Cu site for a typical fluorescent label is 30-40\acute{Å}. For efficient quenching upon reduction of the Cu site, the fluorescent label should be within this distance of the Cu site. PAz can thus be labelled anywhere on the protein surface since the size of this protein (diameter of approximately 25 \acute{Å}) is less than the Förster radius. The shortest distance that can be achieved, without affecting the partner's docking site of either pAz or NiR, is about 15 \acute{Å}. At this distance, fluorescence quenching by the oxidised type 1 Cu is virtually 100%, providing zero-background detection of the reduced state.

The Förster distance can be tuned to achieve energy transfer to only one of the two type 1 Cu sites in the pAz/NiR docked assembly by appropriate choice of the location of the label on the protein surface, so that one site is well within the Förster radius and the other is not (the two type 1 Cu sites in the docked complex are 15-18 \acute{Å} apart).

The method is not only applicable to proteins that contain a redox-active type 1 Cu-site, but also to other proteins with co-factors that exhibit comparable changes in the absorption spectrum upon a change of redox state or another biochemical variable.

Partner proteins may be labelled with dyes that fluoresce at different wavelengths and that are quenched by different redox acceptor moieties, so that the dynamics between the two redox sites in the docked protein complex may be monitored by dual wavelength detection. Suitable fluorophores for labelling the proteins are common in the art, and have been previously listed in the application.

The analyte is preferably a gas at standard temperature and pressure and is, for instance, O₂, H₂, CO₂, CO, NO or N₂O.

The preferred analyte which is detected in the method according to the invention is oxygen. In this preferred embodiment, the protein is typically a redox enzyme and catalyses the oxidation of a substrate using oxygen bound to the protein. When the protein is tyrosinase, for example, oxygen (the “analyte”) is detected and the (co-) substrate of the enzyme is a monophenol or an ortho-diphenol, for example, tyrosine. The redox enzyme preferably has a binding site for the substrate, which may be the same moiety which binds the analyte, or alternatively a different moiety which has suitable properties for binding the substrate.

The analyte may alternatively be hydrogen. A suitable protein for detecting hydrogen is hydrogenase.

Other suitable analyte-protein combinations include:

ATP—ArsA ATPase (an arsenicum transport protein);

CO/O₂—Cytochrome P450

Glucose—apo glucose oxidase;

Ca²⁺—C-type mannose binding protein;

Decamethonium, edrophonium, ethopropazine, acetylcholine or choline—cholinesterase;

Ca²⁺ or Mg²⁺—Guanylyl Cyclase-activating Proteins; and

Inhaled anaesthetics (e.g. halothane)—G-protein coupled receptors.

In all of these examples, the intrinsic fluorescence of the protein, the tryptophan fluorescence, changes upon the binding of the analyte.

The method of detection according to the present invention may be used to monitor the presence and/or concentration of molecules other than the analyte which affect the binding of the analyte to the moiety. For example, since oxygen carrier molecules such as Hc evolved to respond to the physiological demands of an organism, molecules like lactate, uric acid or ions (e.g. H⁺) change the oxygen binding properties and, thus, (in in vivo measurements) the emission levels in the method according to the invention. These molecules (e.g. “allosteric effectors”) typically bind at a position on the protein distant from the moiety and have a (typically small) effect on the structure of the protein. It has been found that some Hcs are more sensitive than others to allosteric effectors. The use of two different Hcs carrying different fluorescent labels in the same sample allows the determination of a fluorescence intensity ratio. If it is known how the effector changes the O₂ dissociation constant for the more ‘sensitive’ protein, the measurement of the fluorescence response of both proteins provides information regarding the concentration of both O₂ and the effector.

The present invention is not limited to the detection of one analyte. The method may be used to detect two or more analytes in a medium. In this embodiment, two proteins which bind different analytes and each comprising different fluorescent labels are used. The two proteins are preferably excitable at the same wavelength.

Alternatively, two or more proteins may be used in the method of the invention with different dissociation constants for the same analyte. This advantageously increases the concentration range of analyte that may be detected, and the accuracy of the concentration measurements.

In these embodiments, the proteins preferably comprise fluorescent labels that fluoresce at different wavelengths. This allows the proteins to be monitored independently, thereby increasing accuracy and reproducibility.

The fluorescent energy label of the protein of the present invention may be a fluorescent dye on the protein surface. This dye may be covalently attached to a specific protein residue or be an intrinsic property of the protein molecule. Suitable fluorophores for labelling the proteins are common in the art, and include Cy5, Cy3 (Trademark name of dyes from Amersham Biosciences), Alexa Fluor (488, 568, 594 and 647), Tetramethylrhodamine (TMR) and Texas Red, (all obtainable from Molecular Probes, Inc). These may be functionalised either with a maleimide linker for binding to a free thiol group on the protein, or with a succinimydyl ester for binding to a free protein amine group. The reaction may involve oxidative coupling of a cysteine thiol group with a maleimide derivative of Cy5.

A typical method of labelling the protein of the present invention would include the steps of 1) adding bicarbonate to a solution of the protein of the present invention, 2) adding ˜100 μl of protein to the functionalised dye, 3) incubating for one hour, 4) stopping the reaction, 5) incubating for a further 15 minutes and 6) purifying the conjugate on a suitable column using, for example, 0.5M NaCl in water as an eluent. The purifying step ensures that most of the proteins become labelled with a dye molecule, thereby increasing the sensitivity of the method. The concentration of protein used according to the present invention should be high enough to allow detection of fluorescence, preferably 0.01 to 10 μM, more preferably 1 to 2 μM.

Typically the fluorescent label is conjugated to a cysteine, lysine or arginine residue or the N-terminus of the protein, optionally through a linker, such as N-hydroxysuccinimide. The label position can be varied by replacing a solvent-exposed residue by a cysteine residue. A label can then be specifically attached to this residue using a sulfhydryl reactive label (usually containing a maleimide reactive group), as detailed in WO 2006/006977.

Alternatively, the protein used in the invention may be intrinsically fluorescent, such as the Aequora-related green fluorescent protein. Fluorescent proteins whose amino acid sequences are either naturally occurring or engineered by methods known in the art are included within the scope of the invention. Fluorescent proteins can be made by expressing nucleic acids that encode fluorescent proteins, such as wild-type or mutant Aequorea green fluorescent protein, in an appropriate cellular host.

The method of the present invention may further comprise a step of relating the emission from the fluorescent label to substrate (or analyte) turnover. For instance, during a typical oxidation reaction, oxygen (the “analyte”) is consumed and therefore the emission from the labelled protein in the invention increases. The rate of increase of emission can be correlated with oxygen (O₂) consumption and therefore also substrate (e.g. glucose) turnover, according to the stoichiometric relationship between the substrate and O₂ in the reaction mechanism.

The immobilised protein used in the method of the present invention may be added to a biological sample before being subject to incident radiation. This may advantageously allow the metabolic rate of the biological sample to be determined. There is no need for macroscopic mechanical interfacing as with the O₂ measurement systems of the prior art. Proteins such as the type-3 copper proteins have evolved to selectively bind oxygen in a biological setting, with minimal interference from other compounds. Oxygen binds reversibly and no O₂ is consumed during the method.

Typically, the biological sample is from an animal or human, typically animal or human tissue, more typically muscle, nerve or brain tissue. Respiration occurring in the biological sample consumes oxygen (the analyte) resulting in an increase over time in fluorescence from the sample. The protein may be added to the sample either in vivo (for example by direct injection into the tissue) or ex vivo (the protein is added to tissue obtained from cell cultures or from tissue following extraction of the tissue from the animal or human body).

As oxygen metabolism is elementary to all O₂ respiring organisms, its quantification in biological samples is highly valuable. The O₂ consumption is a direct measure of metabolic rate, which in turn is a measure of the activity of living cells.

The metabolic rate is also an important parameter in drug-screening, i.e. the screening of large libraries of drug candidates using mass in vitro diagnostics. O₂ consumption is a parameter of cell viability (with or without drugs added) to interrogate drug candidates for toxicity properties.

The encapsulated protein may be immobilised on a carrier. Preferably, the carrier is an electrode, particularly when the protein is a redox enzyme. For instance, the polymer matrix with the protein encapsulated therein may be coated onto a carrier, e.g. an electrode. Associating the labelled protein with electrodes allows direct electron transfer between the protein and the electrodes. This offers potentiostatic control over the redox state of the surface layer, and the possibility to perform scanning voltammetry while detecting the fluorescence intensity.

### Matrix

The protein is encapsulated in a biocompatible, optically transparent matrix which is permeable to the analyte. By “biocompatible”, we mean a matrix which does not denature the protein. The matrix should be biologically and chemically inert. The matrix should be transparent to the incident electromagnetic energy (typical wavelengths in the range 250 to 800 nm) which is used to excite the protein or label, and should also transparent to the fluorescent emission to allow its detection. The matrix should allow diffusion of the analyte from the medium suspected of containing the analyte to the protein immobilised in the matrix. The medium may either be a gaseous or an aqueous medium.

The matrix is generally a polymer matrix.

Two requirements for an optically based oxygen sensor are: a) transparency and b) inertness of the matrix chosen for the immobilization. These requirements are met by silica based matrices, which, especially in the last decade, have become an established tool for enzyme encapsulation giving rise to biocatalysts that can be easily recycled [6]. By tuning the polymerization reaction conditions (e.g. pH) these so-called sol-gel materials can be designed for a given specific application meaning that the gels can be tailored to a range of porous textures, network structures, surface functionalities and processing conditions. Furthermore, the manufacture of the sol-gel does not require harsh reaction conditions which is an advantage when working with the often delicate proteins that have to be incorporated in the matrix. It allows proteins to retain their native structure, spectroscopic properties and (catalytic) activity upon encapsulation into the matrix.

Preferably, the matrix is a sol-gel matrix, more preferably a silica sol-gel matrix. Suitable sol-gel matrices are further described in references 6, 8 and 9. In particular, sol-gel matrices made from tetramethylorthosilicate (TMOS), tetraethylorthosilicate (TEOS) and/or Si(OCH₃)₄ are preferred. Sodium metasilicate (Na₂SiO₃) may also be used.

In an alternative embodiment of the invention, organic matrices, such as those derived from polyvinyl alcohol (PVA) may be used. Example 5 gives instructions for making a suitable PVA solution for protein immobilisation.

Techniques for immobilising proteins into matrices, particularly sol-gel matrices are well known. Reference 8 describes suitable techniques.

Preferably, a layer of immobilised protein in the matrix is coated onto a support, such as a glass of quartz slide. Typically, the layer of matrix has a thickness in the range 0.1-2 nm, although for some applications the thickness can be less than 100 nm.

The above methods of immobilisation advantageously provides high concentrations of the protein molecule. This may be advantageous, for example, in in vivo confocal microscopy.

The method may be performed in a kit comprising an optical set-up that makes use of total internal reflection to excite a layer of fluorescently labelled protein molecules immobilised on electrodes. The electrodes are mounted in an optical microscope equipped with laser excitation and a high aperture objective to monitor the fluorescence emitted from the protein coated on the electrode. The electrodes are transparent to light of wavelength for exciting the fluorescent protein residues and to the fluorescence emitted by the label. The protein is preferably an enzyme. In addition, a three electrode electrochemical set-up may be connected to the sample compartment and the electrode immersed in buffer to which enzyme substrate can be added. The enzyme may be regenerated either by a voltage sweep or chemically by making the electrode part of the flow cell and directing a redox active flow over the electrode.

### BRIEF DESCRIPTION OF THE FIGURES

FIG. 1 illustrates the principle of the FRET based O₂ sensing;

FIG. 2(A) shows the absorption spectrum of oxygenated Ty corrected for the contribution of the reduced protein between 300 and 500 nm (main panel) and between 500 and 850 nm (inset), and fluorescence emission spectra of fully reduced Ty and fully oxygenated Ty (dashed line) upon excitation at 280 nm;

FIG. 2(B) is the absorption spectra of the dyes Alexa350, Atto390, Cy3, Cy5 and Atto655 and their overlap with the Ty tryptophan emission (dashed line);

FIG. 3A shows the distances between Trp and the terminal-N (label attachment point; dark panels) and between Trp and the closest Cu of the type-3 center (light panels) in S. antibioticus Ty as modelled on the S. Castaneoglobisporus structure [7];

FIG. 3B shows the excitation spectrum of Ty-Cy5 and the free Cy5 dye at a detection wavelength of 665 nm;

FIG. 4A shows the label emission of different Ty-label conjugates of the oxygen-free, reduced protein (solid lines) and of the oxygenated form (dashed lines) upon excitation at 280 nm;

FIG. 4(B) shows the reversible oxygenation and deoxygenation of a solution containing a mixture of unlabeled Ty (95%) and Ty conjugated with Cy3 (5%);

FIG. 5(A) shows the titration results of Hc-Atto390 with O₂ monitored by the dye emission upon excitation at 280 nm;

FIG. 5B shows the emission spectra (λₑₓ=280 nm) recorded for a mixture of BSA-Atto390 and Hc-Cy5 containing various [O₂];

FIG. 6A shows the absorption spectrum of oxygenated (black) and oxygen-free (grey) Octopus vulgaris Hc and the CT band around 570 nm (inset; oxy: black; deoxy: grey);

FIG. 6B shows the spectral overlap of the 570 nm band of O.v. Hc (black) with the emission spectrum (λem=665 nm) of Cy5 (grey). Protein concentration: 2.1 mM;

FIG. 7 shows reversible oxygenation and deoxygenation of a solution containing Hc labelled with Cy5 (2.4 μM). Grey trace: excitation of the dye through the Trp emission (λeₓ 295 nm); black trace: direct excitation of Cy5 at 645 nm;

FIG. 8 shows absorption spectra of two undoped sol-gels on a quartz support; grey: waterglass based; black: TMOS based. Thickness of the sol-gels was around 0.6 mm;

FIG. 9 shows fluorescence spectra of two undoped sol-gels on a quartz support upon excitation at 295 nm; grey: waterglass based; black: TMOS based. Thickness of the sol-gels was around 0.6 mm; and

FIG. 10 shows fluorescence spectra of two undoped sol-gels on a quartz support upon excitation at 645 nm; grey: waterglass based; black: TMOS based. Thickness of the sol-gels was around 0.6 mm; and

FIG. 11 shows the fluorescence intensity as a function of time for hemocyanin Cy5 upon deoxygenation and oxygenation; excitation at 295 nm (grey) and 645 nm (black); protein concentration 5-10 μM. The sample was immersed in a gaseous atmosphere. A: waterglass based sol-gel; B: TMOS based sol-gel.

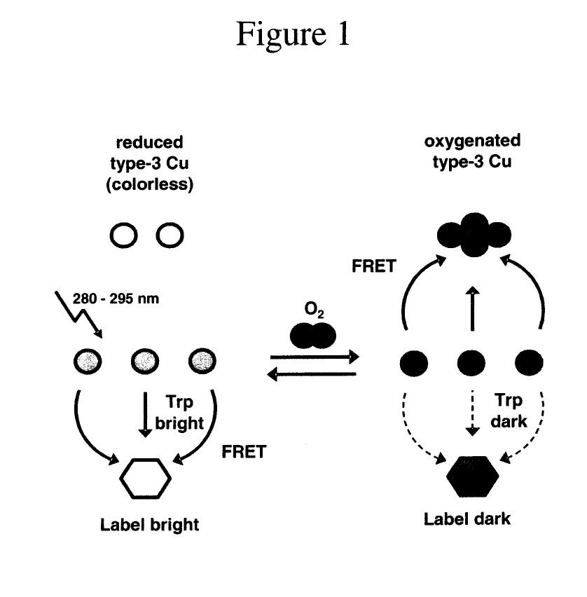

The invention will now be illustrated by the following Examples.

### Example 1

### Protein Trp Fluorescence and O₂ Binding (Reference)

To illustrate this Example, Ty from the soil bacterium Streptomyces antibiotius was selected.

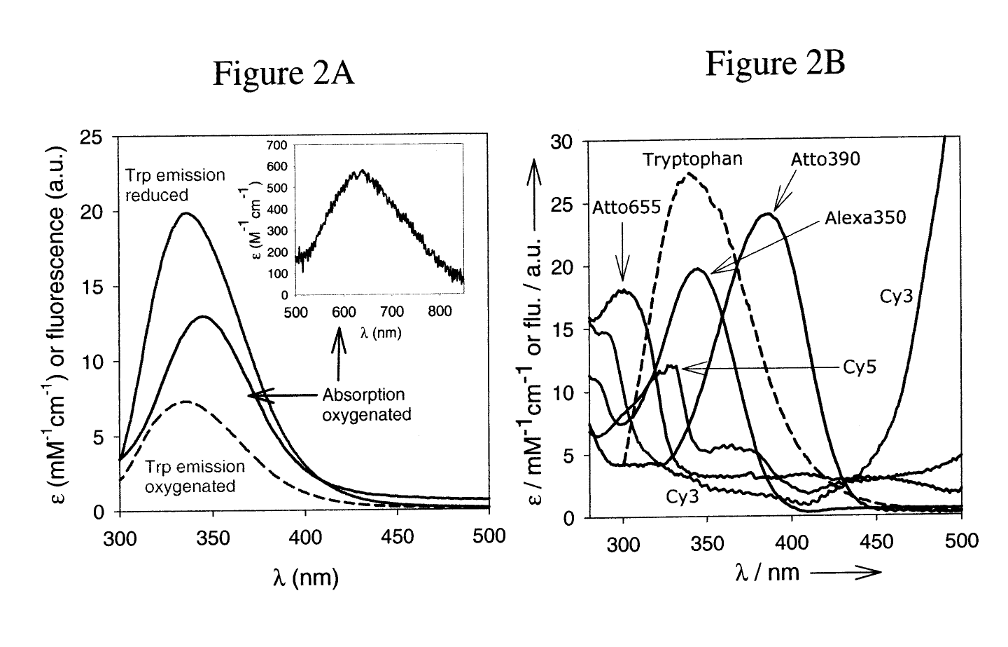

S.c. Ty contains 12 Trp residues on a total of 271 amino-acids (4.4% against ˜1% on average [10]), which cluster around the type-3 centre. The Trp fluorescence shows a maximum at 339 nm upon excitation at 280 nm. In an air-saturated solution (0.26 mM O₂), practically all protein occurs in the oxygenated form (see FIG. 2A). Upon complete deoxygenation, the Trp fluorescence increases by a factor of 2.7 while the shape and position of the emission band remain unchanged. For the C.a. Hc samples, this factor, further referred to as the switching ratio SR (=F_{red}/F_{oxy}), amounts to 2.2.

### Example 2

### Dyes (Reference)

FIG. 2B shows absorption spectra between 250 and 500 nm of the five dyes selected for this study: Alexa350, Atto390, Cy3, Cy5 and Atto655, as well as their overlap with the Ty tryptophan emission. The excitation wavelength was 280 nm. Together these dyes emit at wavelengths spanning the whole visible spectrum (Table 2 and FIG. 2B). This allows the researcher to choose a dye which emits at a wavelength which does not interfere with other fluorescent systems in the sample.

TABLE 2
Switching ratios, spectral overlap integrals and Förster radii for
Trp and the dyes utilised in this study.

| | | | | | Ro, Trp |
|---|---|---|---|---|---|
| | λem | SR^{[a]} | SR^{[a]} | Jₜᵣₚ Ty^{[b]} | Ty^{[c]} |
| Dyes | (nm) | Tyr | Hc | (nm⁴M⁻¹ cm⁻¹ × 10⁻¹³) | (Å) |
| Trp | 339 | 2.7 | 2.2 | 13.0 | 23 |
| Alexa350 | 440 | 1.8 | 2.4 | 16.5 | 24 |
| Atto390 | 470 | 2.2 | 2.1 | 21.1 | 25 |
| Cy3 | 566 | 2.8 | 2.2 | 5.9 | 20 |
| Cy5 | 665 | 4.2 | 2.3 | 5.8 | 20 |
| Atto655 | 684 | 4.3 | 2.1 | 7.2 | 21 |

^{[a]}denotes the dye emission switching ratio (F_{red}/F_{oxy}) observed with excitation at 280 nm.
^{[b]}Calculated spectral overlap integrals between the Trp emission and the absorption of the oxygenated protein (for Trp) or between the Trp emission and the absorption of the dye (for the labels).
^{[c]}Förster radii R₀ were calculated using φ = 0.07, k² = ⅔ and n = 1.4. [11].

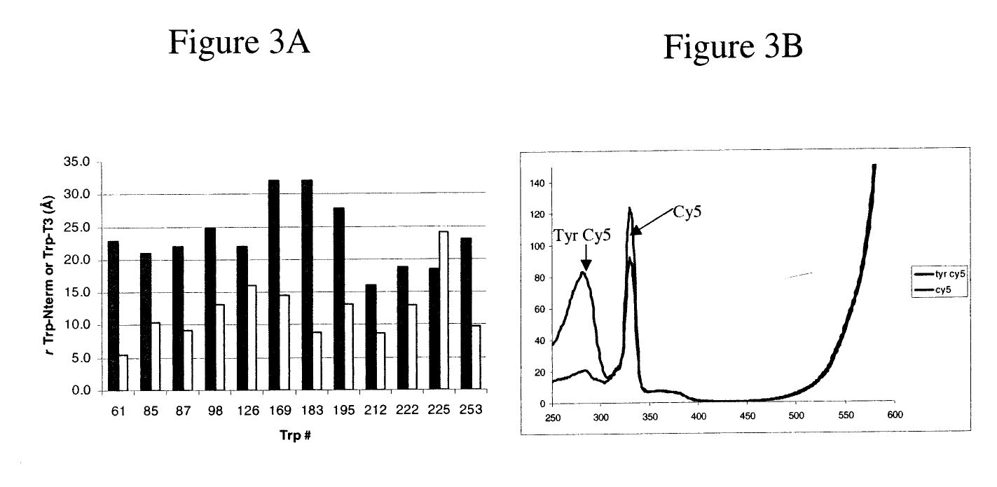

For Ty the spectral overlap integrals of the dye absorption bands with the Trp fluorescence spectrum have been collected in Table 2 together with the corresponding R₀ values. The latter are all in the range of the distances between the Trp residues and the amino-terminus (16-32 Å), which is the attachment point of the label (FIG. 3A and Table 3). This confirms that for Ty FRET from Trp to the selected labels will be observable. The Trp emission spectrum of Hc is similar to that of Ty and yields similar overlap integrals and R₀ values (not shown).

TABLE 3
Distances between Trp and the terminal-N and between Trp and
the closest Cu of the type-3 centre in S. Antibioticus Ty.

| Trp | rTrp-Nₑᵣₘ | rTrp-T3 |
|---|---|---|
| residue # | (\acute{Å}) | (\acute{Å}) |
| 61 | 22.8 | 5.5 |
| 85 | 21.0 | 10.4 |
| 87 | 22.1 | 9.3 |
| 98 | 24.7 | 13.1 |
| 126 | 22.1 | 16.1 |
| 169 | 32.0 | 14.5 |
| 183 | 32.2 | 8.9 |
| 195 | 27.9 | 13.1 |
| 212 | 16.1 | 8.7 |
| 222 | 18.8 | 13.0 |
| 225 | 18.4 | 24.0 |
| 253 | 23.0 | 9.8 |
| Average | 23.4 | 12.2 |

### Example 3

### Reference

### O₂ Binding and Label Fluorescence

Excitation absorption spectrums were recorded in this Example using a monochromator in the detection path, that has its wavelength set at an emission band of the molecule to be studied, in this case 660 nm for Cy5. Excitation is achieved by using a white light source and employing a second monochromator between light source and sample. The wavelength of this monochromator was slowly scanned. When the wavelength of the excitation light matched an absorption band of Cy5, the dye started to fluoresce (at 660 nm) and the emitted light was observed in the detection set-up. By recording the fluorescence intensity as a function of the wavelength of the exciting light, the absorption spectrum was obtained.

Both Hc and Ty were labelled at their N-terminus with each of the five dyes [12]. When setting the detection wavelength at the dye emission maximum, the excitation spectra of the labelled proteins exhibit a strong peak at 280 nm, i.e., the wavelength of the Trp absorption (see FIG. 3B for Ty-Cy5). The peak around 330 nm is a second-order artefact. In a solution containing dye and protein separately (not linked) this peak (280 nm) was missing. The second order artefact is due to higher orders of light being allowed through the monochromator. The artefact can be excluded by placing a cut-off filter in the detection path that allows through light only beyond a certain wavelength (for example 600 nM).

These observations confirm that in the labelled proteins FRET from the Trps to the label occurs. The detection limit of the weakly fluorescent conjugates carrying a Cy3, Cy5 or Atto655 label was around 1 nM using standard equipment. The spectra (FIG. 3B) have been normalised to the emission intensity of Cy5 (excitation at 645 nm; detection at 665 nm).

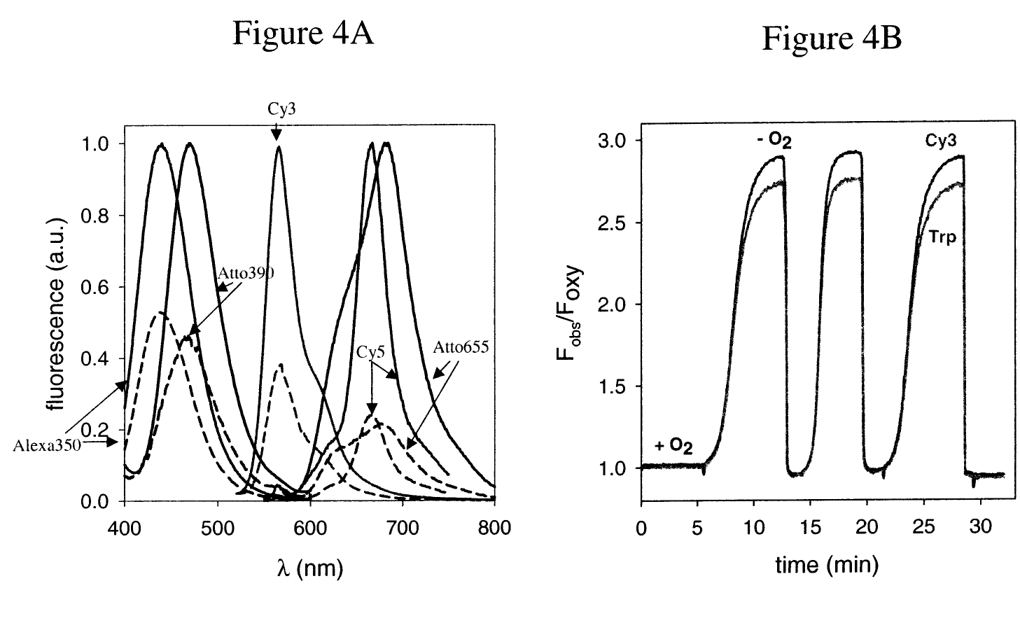

In all cases, an increase in label emission was observed after deoxygenating the sample (FIG. 4A). This process was fully reversible as illustrated in FIG. 4B for a sample containing a mixture of unlabeled Hc (95%) and Hc-Cy3 (5%). The Figure shows Trp and label emission intensities as a function of time while the sample is repeatedly deoxygenated and oxygenated. The Cy3 fluorescence is specific for the labelled protein, while the Trp emission derives mainly (>95%) from unlabelled Hc. Similar observations were made for other labels, and also when Ty was used instead of Hc. The Trp and the label emissions vary in a similar manner. Additionally, the presence of the label does not affect the K_{d} for O₂ binding of the proteins.

### Control

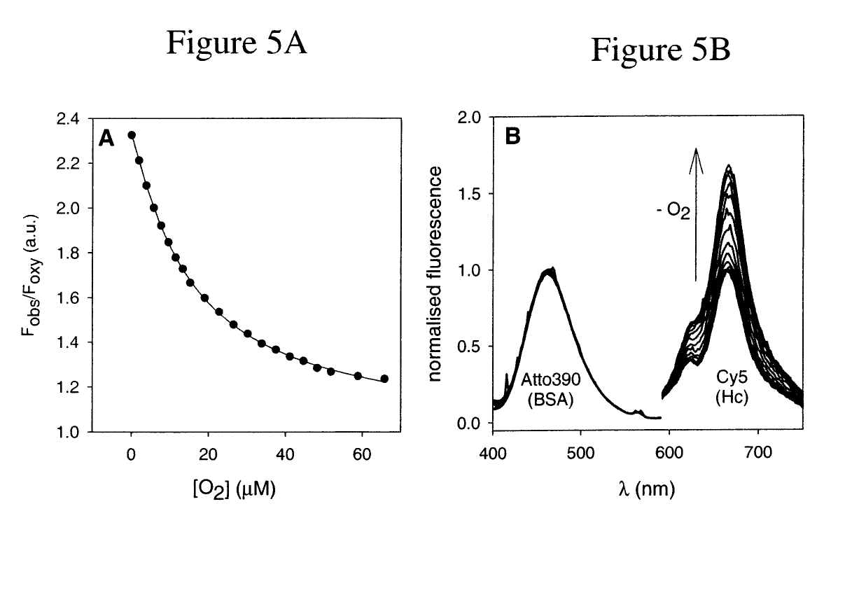

To rule out that the changes in dye fluorescence might be due to a specific quenching by molecular oxygen, a mixture of Atto390 labelled bovine serum albumin (BSA) and Hc-Cy5 was deoxygenated while recording emission spectra from 300 to 750 nm upon excitation at 280 nm. The applied labels emit at different wavelengths, therefore permitting the simultaneous observation of each individual conjugate. The emission of Hc-Cy5 displays a clear dependence on [O₂], while the emission of BSA-Atto390 does not (FIG. 5B). With the labels exchanged, the reverse was observed. From this it can be concluded that the variations in label fluorescence correlate with the binding of O₂ to the labelled hemocyanin. Again, the fluorescence of the isolated dyes does not show a dependence on [O₂]. Similar observations were made for Ty.

### Cooperative or Non-Cooperative Oxygen Binding of Labeled Hc

Hcs, in the native multimeric forms, display cooperative O₂ binding due to allosteric interactions between the constituent subunits of the Hc. A titration of Hc carrying the Atto390 label with O₂ monitored by following the label fluorescence (FIG. 5A) showed that this cooperativity is lost. The solid line in FIG. 5A represents the best fit to the data. The titration data were fitted to a modified form of the Hill equation:

$${F}_{[ {O}_{2} ]} = {F}_{red} - \frac{( {F}_{red} - {F}_{oxy} ) \cdot {[ {O}_{2} ]}^{n}}{{[ {O}_{2} ]}^{n} + {K}_{d}} \qquad ( Equation 1 )$$

This resulted in a dissociation constant K_{d} of 22 μM and a Hill constant n of 1.09, diagnostic of almost total loss of cooperativity. It is known that arthropod Hcs dissociate into the subunits at high pH and in the absence of divalent metal ions. Since both conditions were met during the labelling procedure, it is likely that our Hc samples contain a mixture of dissociated labelled subunits. The Ty, in contrast, is monomeric and also does not show cooperative O₂ binding in the native or labelled forms (vide infra).

### Discussion

### Switching Ratios

A few nanomolars of construct could be easily detected with a standard fluorimeter. Next to a good sensitivity, a high contrast in label fluorescence between O₂-free and O₂-bound protein is crucial for practical sensing applications. For the Hc, the dye switching ratio SR (=F_{red}/F_{oxy}), is similar to the SR value for the Trp fluorescence of the native protein (Table 2). For Ty, the SR values seem to vary between the labels. Very high contrast may be achieved by considering carefully the relative positions and orientations of the label, the type-3 centre and the fluorescent Trp residues.

### Example 4

Experiments performed in bulk solution (Example 4.1) are described first. These provide a reference for judging the sol-gel samples. The sol-gel experiments are dealt with in the next section (Example 4.2). Example 4.3 describes a number of controls.

### 4.1. Bulk Experiments

Initially three different hemocyanins, (Hcs), (from the arthropod Carcinus aestuarli and from the molluscs Rapana thomasiana and Octopus vulgaris) were selected and labeled with various dyes at their N-termini, as described elsewhere [13]. The dye switching ratio (SR) is defined by SR=(F_{deoxy}−F_{oxy})/F_{deoxy}, in which F_{oxy} and F_{deoxy} are the emission intensities of the fully oxygenated and the fully deoxygenated protein upon direct excitation of the fluorophor. They have been determined for all three Hcs in solution (Table 4). The SR was measured also for the tryptophan residues of each Hc (unlabeled) (Table 4). In two similar studies the SRs for Trp (92%) and the attached dye TAMRA-SE (50%) have been measured for Hc of the tarantula Eurypelma californicum [14, 11].

TABLE 4
Switching ratios (SR, expressed as percentage) obtained in bulk
for three different hemocyanins upon direct excitation of the dye at the
wavelength of maximum absorption (λ_{abs}).

| | λ_{abs} | λₑₘ | SR Hc | SR Hc | SR Hc |
|---|---|---|---|---|---|
| Dye | (nm) | (nm) | Carcinus | Octopus | Rapana |
| Alexa350 | 346 | 440 | 19% | 44% | 54% |
| Cy3 | 550 | 570 | 22% | 40% | 53% |
| Cy5 | 645 | 665 | 33% | 55% | 23% |
| Atto655 | 663 | 684 | 35% | 42% | 13% |
| Alexa568 | 580 | 602 | 33% | 53% | 57% |
| Trp | — | 334 | 80% | 58% | 78% |

In further experiments we focused on hemocyanin from Octopus vulgaris (O.v.) labeled with Cy5. The typical architecture of an Octopus hemocyanin, which consists of a string of seven globular functional units [15, 16] (designated a to h, about 50 kDa each), was verified on a 5% SDS-page gel (data not shown).

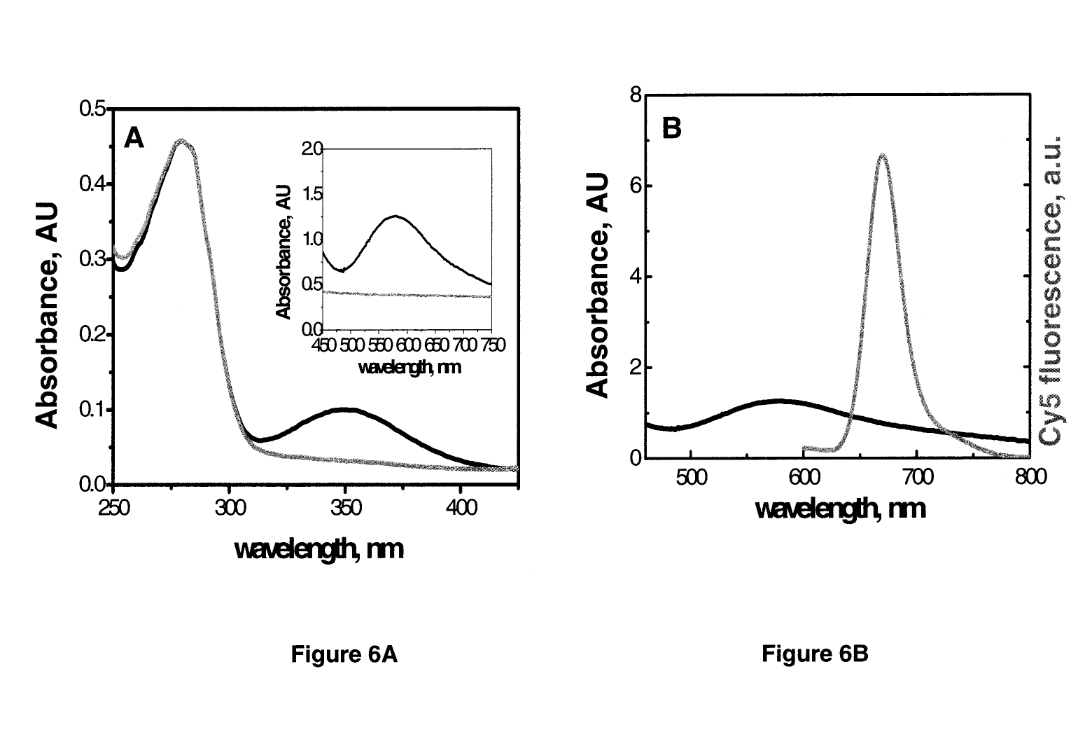

In order to check the quality of our samples, we recorded the spectroscopic features of this protein (for details see the Experimental section). FIG. 6A shows the absorption spectrum of (O.v.) Hc in the oxygenated and the deoxygenated state and the CT band around 570 nm (inset). The overlap of this band with the emission spectrum of Cy5 can be judged from FIG. 6B. In the Hc deoxygenated state the 570 nm CT band is missing. The difference in spectral overlap modulates the quenching of the label fluorescence.

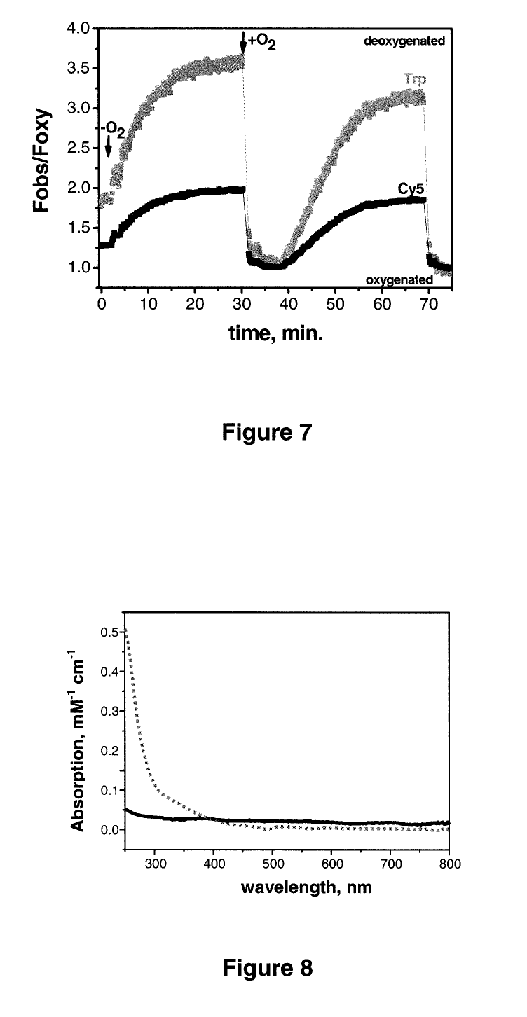

The previously reported ‘sensitised fluorescence approach’, where the dye, attached to the protein, is excited by the emission of endogenous Trp residues, can also be observed with this construct. An oxygenation/deoxygenation time-trace was recorded by exciting at two wavelengths alternatingly (with ˜5 second intervals) at the dye absorption maximum (see Table 4) and at the absorption maximum of the tryptophan (295 nm). The dye emission was followed at 665 nm. FIG. 7 shows typical traces of the dye response. A switching ratio of approximately 50±5% for the direct excitation of Cy5 and of 70±5% for the excitation through the tryptophans was observed. The SR observed by exciting at 295 nm is in accordance with our previous findings [13].

Taking into account the theoretically estimated R₀ value of 2.7 nm in the oxygen bound state a FRET efficiency of roughly 0.56 for direct excitation of Cy5 is predicted, which is in good agreement with the experimental value (for further details see experimental section).

The complete removal of [O₂] from the sample, by passing Argon through the solution, takes around 30 minutes. The fraction of O₂-free protein changes accordingly. Oxygenation occurs in a much shorter time period, which is related to the relatively low K_{d} for oxygen (22 μM). From FIG. 7 it is clear that the minimum and maximum levels of the fluorescence decrease with increasing number of cycles. We ascribe this to damage inflicted to the Hc by the bubbling of the Ar or oxygen through the solution. For the sol-gel encapsulation experiments dealt with in the next section we focused again on the Cy5 O.v. hemocyanin construct.

### 4.2. Sol-Gel Matrices

### Undoped Sol-Gels.

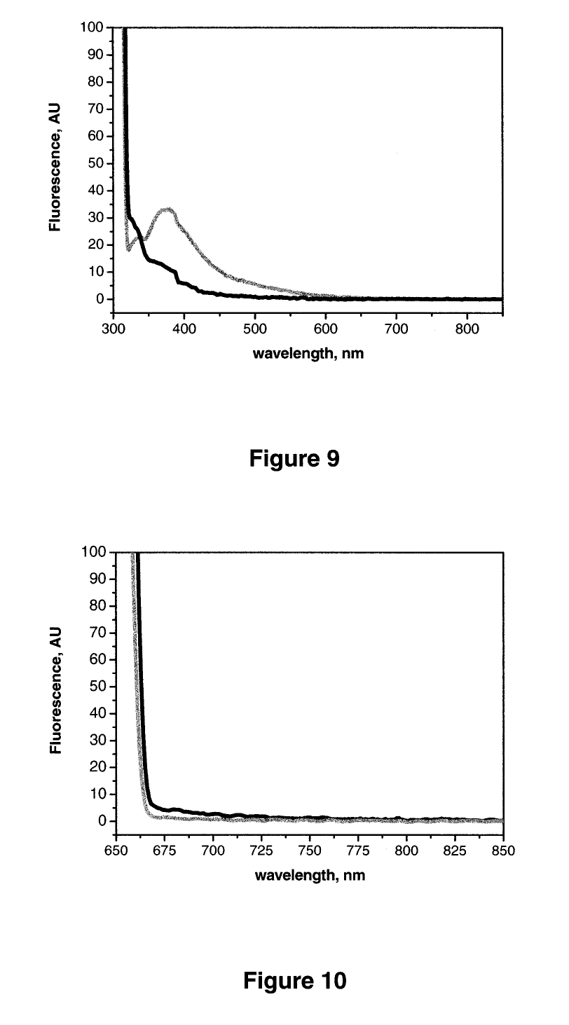

Absorption spectra of the undoped sol-gels (TMOS- (black) and waterglass-based (grey) as described in the experimental section, thickness about 0.6 mm) were measured on a quartz support and confirmed the transparency of the samples in the 250-800 nm range (FIG. 8). Fluorescence from the sol-gel about 0.6 mm thick, upon excitation at 295 nm (FIG. 9) and at 645 nm (FIG. 10), was virtually absent for the TMOS case (black); for the waterglass-based sample (grey) a weak band around 380 nm was obtained when excited at 295 nm. In all cases the emission intensity observed is negligible in comparison with that of the doped samples (see below).

### Hc Doped Sol-Gels.

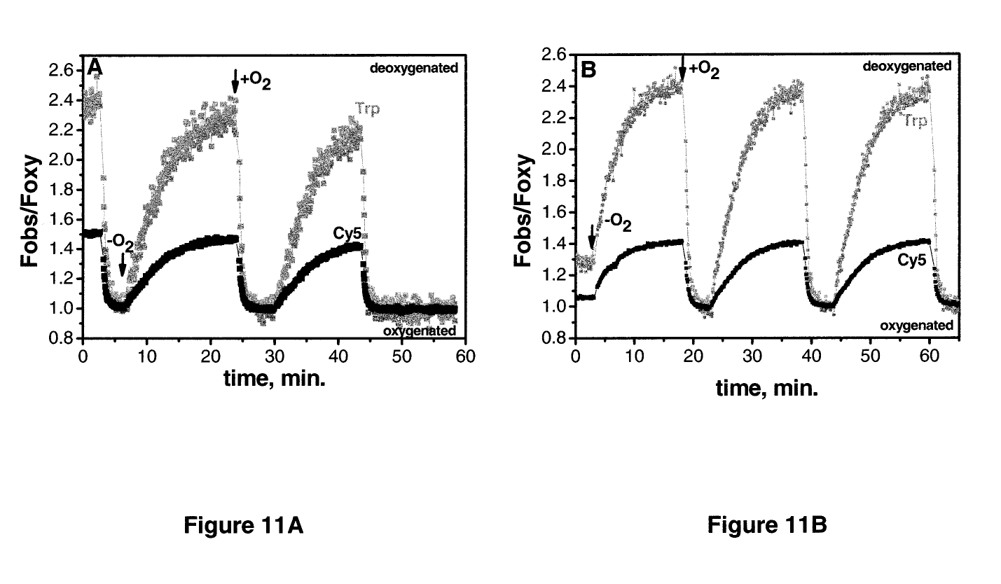

The fluorescence intensity of the encapsulated Cy5-labeled Hc was monitored as a function of time during the change from oxygen saturated to an oxygen free environment and vice versa for a) the sample immersed in a gaseous atmosphere and b) the sample immersed in solution. Two parameters were determined: a) the SR and b) the time response as described in the previous section. The first two time-traces shown in FIG. 11 were measured on two different samples in a gaseous surrounding, one waterglass- (A) and the other TMOS-based (B).

In the gas phase the deoxygenation response time (about 16 mins) for both sol-gels (waterglass and TMOS) appears to be twice as short as in solution (about 32 minutes). This probably reflects the faster diffusion of the oxygen in a gaseous atmosphere than in solution.

Concerning the SRs, values of roughly 30% for the direct and circa 60% for the indirect excitation of the dye were observed both for the water glass- and the TMOS-based sol-gels. These values are smaller than the ones obtained in the bulk. Possibly a fraction of encapsulated enzyme is not accessible to the substrate [6] or has been damaged and lost its capability to bind O₂. This would increase the background fluorescence and cause a decrease of the SR.

For the measurements of TMOS- and waterglass-based samples in surrounding buffer, SRs of 25% in case of direct excitation of the dye and 50% for excitation through the Trps were obtained (data not shown). The response time was 32 minutes for both sol-gels, comparable to the bulk experiments (30 minutes). The ‘shelf life’ of the samples was found to be at least 15 days upon storage at 4 degrees as judged by the lack of decrease in either the SR or the response time of the samples.

### 4.3. Controls

Several experiments were performed as controls: 1) The fluorescence intensity of neither unreacted Cy5 nor bovine serum albumin labeled with Cy5, both individually encapsulated in a sol-gel matrix, depended on the [O₂]. [19]. The buffer solution surrounding the quartz slides was checked for leaking of the labeled protein out of the matrix. After prolonged incubation of the solgel in a buffer solution (one to two days), the buffer showed no trace of dye fluorescence or absorption. As already reported, the mechanical strength of silica gels often prevents the leaking of biomolecules. 3) Tryptophan fluorescence has been used to study stability and flexibility of proteins upon entrapment [17]. For example, the emission maximum of correctly folded Hc occurs at 331 nm, as reported in [18], while a shift to higher wavelength indicates that initially buried Trp residues become exposed to the solvent, possibly signalling denaturation of the protein. Hence, Trp emission spectra of encapsulated Hc were measured over several weeks. After storing the sol-gels for 7 weeks at four degrees, no significant alterations in the bandshape or intensity of the Trp emission could be detected, suggesting that the enzyme is stable in the matrix.

### CONCLUSIONS

The present data provide proof of principle that the previously published results on ‘sensitized’ fluorescence for oxygen sensing remain valid when labeled hemocyanin is encapsulated in a polymeric sol-gel matrix. It has been reported that TMOS is less favourable than water glass for the immobilisation of biomolecules due to generation of methanol as a side product which might lead to denaturation of the encapsulated protein [19, 20]. In the experiments this appears not to be the case as the same activity is observed in both sol-gel matrices. The relative robustness of the hemocyanin might contribute to this feat. The present implementation of labeled Hc into an oxygen sensor features a fast response, biocompatibility, reusability and outstanding stability. Furthermore, the large variety of Hc types, characterized by different oxygen affinities, provides a broad range of oxygen sensing capabilities.

### Experimental

### General.

Hemocyanin (Hc) from arthropod Carcinus aestuarii and from molluscs Octopus vulgaris and Rapana thomasiana were prepared following the procedures described elsewhere [21]. The purity of the Hcs was checked spectrophotometrically by measuring the ratio of the absorbances at 340 nm and 278 nm and comparing them with literature values (A₃₄₀/A₂₇₈=0.21 (arthropod Hcs) or A₃₄₀/A₂₇₈=0.25 (molluscan Hcs)) [22]. Alexa568 and Alexa350 NHS-ester were from Molecular Probes (Leiden, The Netherlands), Cy5 and Cy3 NHS-ester were purchased from Amersham Biosciences (Freiburg Germany) and Atto655 and Atto390 NHS-ester were purchased from ATTO-TEC Biolabeling and Ultraanalytics (Siegen, Germany). 50 mM stock solutions of the dyes were prepared by dissolving the powders in water-free DMSO. All purification steps during protein labeling were performed using PD-10 gel-filtration columns (Amersham Pharmacia). Sodium silicate, Dowex 50WX8-100 ion-exchange resin and TMOS (tetramethyl orthosilicate) were obtained from Sigma Aldrich.

### Protein Labelling.

Hc was labeled at the N-terminus in potassium phosphate 100 mM, pH 6.8, using procedures reported in literature [3]. Labeling ratios were in the range of 0.2-1 (dye molecule/protein), as determined from the absorption spectra of the labeled proteins using the extinction coefficients of the 280 nm protein absorption [22] and of the labels as stated by the manufacturers respectively.

### Protein Encapsulation in TMOS Sol-Gel

The preparation of silica gels and the encapsulation of proteins were undertaken with pure TMOS, according to a previously reported procedure [8]. TMOS (15.22 g) was mixed with milliQ water (3.38 g) in a 1:2 molar ratio followed by the addition of 20 μl of 10 mM HCl. The reaction mixture was sonicated for 20 minutes. Upon addition of buffer (potassium phosphate 100 mM, pH 6.8) in a 1:1 (volume) ratio and roughly 1 ml of labeled protein solution (end conc. of protein: 5-10 μM) the mixture was degassed to remove possible air bubbles. Before gelation 150 μL of the sol solution was quickly poured onto a home made device (quartz slide Heraeus 3 quality with a 1 mm thickness, a 30 mm height and a 8 mm length) yielding a roughly 0.6 mm thick sol-gel layer on top of the quartz slide. Activation of quartz slides with “Piranha solution” (30% H₂O₂ and concentrated H₂SO₄ in a 1:3 volume ratio) was performed before pouring the sol solution on top. Samples were kept sealed with parafilm and aged at 4° C. overnight before the measurements. Methanol, a byproduct of the TMOS-based sol-gel process, was removed by washing the samples with potassium phosphate buffer (100 mM, pH 6.8) several times.

### Protein Encapsulation in Sodium Silicate Sol-Gel.

Sol solution was prepared as described previously [9]. Sodium silicate solution (0.83 ml) was mixed with milliQ water in a 1:4 (volume) ratio and the resulting solution was vortexed. Upon addition of a strongly acidic cation-exchange resin (Dowex 50WX8-100), the pH of the solution was lowered to a value of 7.0. The resin was filtered off by vacuum filtration and the labeled enzyme (end conc.: 5-10 μM) was added, the mixture was degassed to remove the possible air bubbles. Pouring, quartz activation and aging was performed as described above.

### Absorbance and Fluorescence Measurements.

Absorption spectra were recorded on a Cary-50 Spectrophotometer. Fluorescence spectra and time traces were measured on a Cary Eclipse Spectrophotometer by fixing the glass or quartz slides into a home made device in a 10*10 mm airtight quartz fluorescence cuvette (Hellma Benelux by, Rijswijk, Netherlands) and applying front-face illumination [10]. Measurements were performed both in the gas phase and in potassium phosphate buffer (100 mM, pH 6.8) at room temperature. High quality argon (<1 ppm O₂) was blown over the sample or into the surrounding buffer solution to deoxygenate the sample while for oxygenation a flow of pure O₂ was used. In the case of gas phase measurements the gas was humidified by passing it through potassium phosphate buffer (100 mM, pH 6.8) before it was passed into the sample.

Förster Radius Calculations.

R₀ values for FRET from Cy5 to the 570 nm absorption of Hc were determined as previously described [10] from the equation R₀=0.211(Jκ²n⁻⁴Φ_{D})^{1/6} (Å). Here κ₂ is an orientation factor, n—refractive index, Φ_{D}—fluorescence quantum yield of the donor and J—spectral overlap integral, defined as J=∫F_{D}(λ)ε_{A}(λ)λ⁴dλ/∫F_{D}(λ)dλ, where F_{D}(λ) is the fluorescence intensity of the donor, ε_{A}(λ)—the extinction coefficient in [M⁻¹cm⁻¹] of the acceptor at wavelength λ with λ expressed in nanometers. The refractive index was assumed to be 1.4 and the orientation factor κ² was taken to be ⅔. Φ_{D} for Cy5 was taken to be 0.27 [23].

The distance (R) from the Cy5 to the active site was estimated as R=(d+1)nm±0.5 nm (adding 1 nm to the calculated distance d accounts for the approximate length of the linker chain), where d is the distance from the attachment point of the dye (N-terminus). The distance d was estimated to be 2.6 nm from the Hc crystal structure for Octopus vulgaris [24]. The distance (6.2 nm) to the type-3 site of the other oxygen binding sites was too large to contribute to any additional quenching effect. In total 7 tryptophan residues per protomer are surrounding the active site. A quantitative treatment of the quenching of the sensitised fluorescence of the label was not attempted at this stage.

### Example 5

### Directions for Making a Polyvinyl Alcohol Solution

Two methods for making PVA solution are outlined below. The second is taken from reference [25].

### Method 1

1. Heat approximately 1.7 L of distilled water to 60-70° C. Slowly sprinkle 80 g of PVA powder into the hot water with vigorous magnetic stirring. Add slowly so as to avoid clumping of the material.

2. Cover the beaker with plastic wrap and continue heating at 60-80° for 4-6 hours until solution clears. Do not exceed 80° C. The time for dissolution varies considerably from as little as 1 hour to as much as 6 hours.

3. The solution can be diluted while still warm to 2 L. A small amount of residual solid (“scum”) does not seem to affect performance.

### Method 2

1. Sprinkle 40 g of PVA into 1 L of water.

2. Heat the mixture and stir over moderately high heat. If it is still cloudy after an hour of heating, turn up the heat but be careful not to burn the solution.

3. Allow the solution to cool before using. If a slimy or gooey layer appears on the top upon cooling, skim it off and discard. Store in a labelled, closed container, such as a 2 L soda bottle.

A 4% solution of PVA according to one of the above recipes is made up and diluted 1:1 with a buffered protein solution. This is then spin-coated onto a Piranha activated glass slide. The use of the slides then follows the methods for the sol-gel coated methods above.

### REFERENCE LIST

1. Stryer et al (1967) Proc Natl Acad Sci USA 58, 179-726
2. Kenworthy et al (2001) Methods 24, 289-296
3. Tolosa et al (2002) Biotechnol. Bioeng 80, 594-597
4. Hartmann et al (1997) Sensors and Actuators B-Chemical 38, 110-115
5. Erker et al Biochem. Biophys. Res. Comm 324, 893-900
6. Pierre, A. C. (2004) Biocatalysis and Biotransformation 145-170.
7. Gadre et al J. Am. Ceram. Soc., 89 [10] 2987-3002 (2006)
8. Ellerby, L. M., Nishida, C. R., Nishida, F., Yamanaka, S. A., Dunn, B., Valentine, J. S., and Zink, J. I. (1992) Science 1113-1115.
9. Bhatia, R. B., Brinker, C. J., Gupta, A. K., and Singh, A. K. (2000)
Chemistry of Materials 2434-2441.
10. Lakowicz J. R. (1996) Principles of Fluorescence Spectroscopy, Kluwer Academic/Plenum Publishers, New York, Boston, Dordrecht, Moscow.
11. Erker, W., Hubler, R., and Decker, H. (2004) European Biophysics Journal with Biophysics Letters 386-395.
12. Kuznetsova et al (2006) Analytical Biochemistry 350, 52-60.
13. Zauner, G., Lonardi, E., Bubacco, L., Aartsma, T. J., Canters, G. W., and Tepper, A. W. J. W. (2007) Chemistry European Journal, accepted.
14. Erker, W., Sdorra, S., and Basche, T. (2005) Journal of the American Chemical Society 14532-14533.
15. Miller, K. I., Cuff, M. E., Lang, W. F., Varga-Weisz, P., Field, K. G., and van Holde, K. E. (1998) Journal of Molecular Biology 827-842.
16. Lieb, B., Altenhein, B., Markl, J., Vincent, A., van Olden, E., van Holde, K. E., and Miller, K. I. (2001) Proceedings of the National Academy of Sciences of the United States of America 4546-4551.
17. Gadre, S. Y. and Gouma, P. I. (2006) Journal of the American Ceramic Society 2987-3002.
18. Ricchelli, F., Beltramini, M., Flamigni, L., and Salvato, B. (1987) Biochemistry 6933-6939.
19. Coiffier, A., Coradin, T., Roux, C., Bouvet, O. M. M., and Livage, J. (2001) Journal of Materials Chemistry 2039-2044.
20. Yu, D., Volponi, J., Chhabra, S., Brinker, C. J., Mulchandani, A., and Singh, A. K. (2005) Biosensors & Bioelectronics 1433-1437.
21. Bubacco, L., Magliozzo, R. S., Beltramini, M., Salvato, B., and Peisach, J. (1992) Biochemistry 9294-9303.
22. Beltramini, M., Dimuro, P., Rocco, G. P., and Salvato, B. (1994) Archives of Biochemistry and Biophysics 318-327.
23. Mujumdar, R. B., Ernst, L. A., Mujumdar, S. R., Lewis, C. J., and Waggoner, A. S. (1993) Bioconjugate Chemistry 105-111.
24. Cuff, M. E., Miller, K. I., van Holde, K. E., and Hendrickson, W. A. (1998) Journal of Molecular Biology 855-870.
25. Woodward, Linda “Polymers All Around You”, Published by Terrific Science Pr; 1^{st} edition (February 1993).

1. A method of detection of an analyte in which a protein capable of binding the analyte and comprising a fluorescent energy label and an energy acceptor moiety capable of accepting energy emitted by the label or protein by Förster energy transfer (FRET), is exposed to incident electromagnetic energy to excite the protein or label, and the fluorescent emission of the label is measured;
characterised in that the protein is encapsulated in a biocompatible, optically transparent matrix which is permeable to the analyte, and in that the protein undergoes no substantial conformational change during the method;
further characterised in that the energy acceptor moiety has a more active and less active state, which is determined by the presence of analyte, and the emission from the label is indicative of the presence of analyte.

2. The method according to claim 1 wherein the biocompatible, optically transparent matrix is a sol-gel matrix.

3. The method according to claim 2 wherein the sol-gel matrix is a silica sol-gel.

4. The method according to claim 3 wherein the silica sol-gel is formed from silica alkoxide precursors, preferably selected from tetraethyl orthosilicate (TEOS), tetramethyl orthosilicate (TMOS) and Si(OCH₃)₄.

5. The method according to claim 1, wherein the energy acceptor moiety is in its more active state when bound to analyte.

6. The method according to claim 1, wherein the incident electromagnetic energy has a wavelength in the range that excites the fluorescent energy label, and emission from the label is reduced when the energy acceptor is in its more active energy acceptor state.

7. The method according to claim 1, wherein the incident electromagnetic energy excites amino acids or a cofactor in the protein, and emission from the label is reduced when the energy acceptor moiety is in its more active energy acceptor state.

8. The method according to claim 7 wherein the incident electromagnetic energy has a wavelength in the range 260-450 nm, more preferably 280-300 nm.

9. The method according to claim 1, wherein the protein is an enzyme.

10. The method according to claim 9 wherein the analyte is a (co-)substrate, inhibitor or cofactor of the enzyme.

11. The method according to claim 1, wherein the acceptor moiety is a metal ion, a metal ion complex comprising two or more metal ions or an organic cofactor.

12. The method according to claim 1, wherein the protein is an oxygen carrier, preferably hemocyanin.

13. The method according to claim 1, wherein the analyte is a gas at standard temperature and pressure, and is preferably oxygen.

14. The method according to claim 1, wherein the energy acceptor moiety is converted between its more and its less active states via a redox reaction.

15. The method according to claim 14 in which the redox reaction involves a redox partner protein accepting or donating electrons to the protein via docking with the protein.

16. The method according to claim 15 wherein the redox partner protein is capable of oxidising or reducing the analyte.

17. The method according to claim 15 wherein the protein is azurin or pseudoazurin, the partner protein is nitrite reductase, and the analyte is nitrite.

18. The method according to claim 1, wherein the matrix is immobilised on a solid surface, preferably an electrode surface.

19. The method according to claim 18 wherein the electrode is optically transparent and total internal reflection is used to excite the protein or label.

20. A biocompatible, optically transparent matrix in which a protein capable of binding an analyte is encapsulated, wherein the matrix is permeable to the analyte and the protein comprises a fluorescent energy label and an acceptor moiety capable of accepting energy emitted by the label or protein by Förster energy transfer, wherein the energy acceptor moiety has a more and a less active state between the moiety can be converted.

21. A biocompatible, optically transparent matrix according to claim 20 wherein the matrix is a polymer.

22. A biocompatible, optically transparent polymer matrix according to claim 20 having at least one further feature that is selected from the group consisting of:
the biocompatible, optically transparent matrix is a sol-gel matrix;
the energy acceptor moiety is in its more active state when bound to analyte;
the incident electromagnetic energy has a wavelength in the range that excites the fluorescent energy label, and emission from the label is reduced when the energy acceptor is in its more active energy acceptor state;
the incident electromagnetic energy excites amino acids or a cofactor in the protein, and emission from the label is reduced when the energy acceptor moiety is in its more active energy acceptor state;
the incident electromagnetic energy has a wavelength in the range 260-450 nm, more preferably 280-300 nm; or
the protein is an enzyme;
the analyte is a (co-)substrate, inhibitor or cofactor of the enzyme;
the acceptor moiety is a metal ion, a metal ion complex comprising two or more metal ions or an organic cofactor;
the protein is an oxygen carrier, preferably hemocyanin;
the analyte is a gas at standard temperature and pressure, and is preferably oxygen;
the energy acceptor moiety is converted between its more and its less active states via a redox reaction;
the matrix is immobilised on a solid surface, preferably an electrode surface;
the electrode is optically transparent and total internal reflection is used to excite the protein or label.

23. A method of making an electrode for detecting an analyte in which a coating comprising a matrix and a protein is coated onto an electrode substrate, wherein the matrix is as defined in claim 20.

24. A method according to claim 23 in which the matrix is a silica sol-gel and is formed by coating silica alkoxide gel precursors, selected from tetraethyl orthosilicate (TEOS), tetramethyl orthosilicate (TMOS) and Si(OCH₃)₄.
